(19) United States
(12) Patent Application Publication
(10) Pub. No.: US 2022/0330549 A1
(43) Pub. Date: Oct. 20, 2022

(54) USE OF N-BENZYLBENZAMIDE-BASED COMPOUND AS HERBICIDE

(71) Applicant: Northwest A&F University, Xianyang City, Shaanxi (CN)

(72) Inventors: Zhiqin JI, Xianyang City, Shaanxi (CN); Shaopeng WEI, Xianyang City, Shaanxi (CN); Huixia ZHANG, Xianyang City, Shaanxi (CN)

(21) Appl. No.: 17/423,247

(22) PCT Filed: Sep. 2, 2020
(86) PCT No.: PCT/CN2020/113018

§ 371 Date: Jul. 15, 2021

(30) Foreign Application Priority Data
Mar. 11, 2020 (CN) 202010163988.9

(51) Int. Cl. A01N 43/40; A01N 37/18; A01N 33/04; A01P 13/00; A01N 65/08

(52) U.S. Cl. CPC A01N 43/40; A01N 37/18; A01N 33/04; A01P 13/00; A01N 65/08

(57) ABSTRACT

An N-benzylbenzamide-based compound as shown below or its salt which may be used as a herbicide is provided.

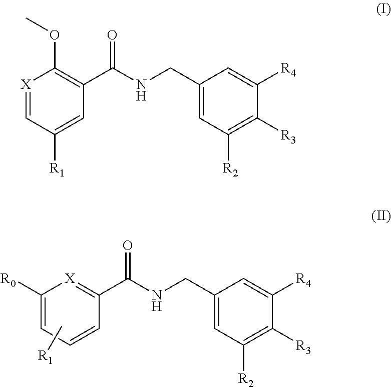

The N-benzylbenzamide-based compound or its salt serves as a pigment synthesis inhibitor for controlling weeds in field crop growing places or non-farming crop places. Furthermore, the compound may form an agricultural herbicide with an agriculturally accepted carrier. Still further, the compound also serves an agricultural herbicide in a manner of a mixed formulation with a further herbicidal active component, particularly atrazine. The N-benzylbenzamide-based compound or its salt has high herbicidal activity, and can be used to control weeds that are resistant to existing herbicides, which thereby exhibits a good synergistic effect when combined with a further herbicidal active component.

### TECHNICAL FIELD

The present application relates to the field of agricultural chemicals, and in particular to the use of an N-benzylbenzamide-based compound as a herbicide. The present application further provides a herbicidal composition and a mixed formulation comprising the N-benzylbenzamide-based compound, and applications thereof.

### BACKGROUND

As a large agricultural country, our country has a large area of farm crops such as wheat, corn, rice, etc., as well as commercial crops such as coffee, tea, rubber, and fruit trees. In field or a non-farming crop places, weeds are common and various, and may compete with the crops for water, nutrients, light and space, which thereby causes a severe reduction in the yield. Although chemical weed control is economical, effective and labor-saving, most herbicides have a limited weed controlling spectrum, and the weed may become herbicide-resistant if adopting the chemical weed control for a long term. Therefore, it is an inevitable trend of future development to research and develop green agricultural herbicides with higher stability, stronger herbicidal activity, wider weed controlling spectrum and less toxic side effects. In addition, prior studies have found that mixing herbicides with different mechanisms of action can effectively improve the weed control effect and weed controlling spectrum, and delay the emergence of herbicide resistance.

Document (1) (Polyhedron, 2017, 129:97-104) reported a method for preparing a compound 1 and the antiviral activity thereof. Prior studies have confirmed that the N-benzylbenzamide-based compound is a compound with excellent anti-tubercle bacilli activity (ACS Med. Chem. Lett. 2018.9(7):741-745). Patent CN2016109689860 provides a variety of N-benzylbenzamide-based compounds having specific chemical structures, which may serve as pharmaceutical ingredients for anti-tuberculosis.

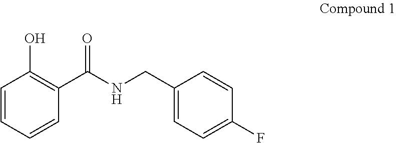 [SMILES: O=C(NCc1ccc(F)cc1)c1ccccc1O]

In the agricultural field, patent EP0467473A1 discloses the herbicidal activity and a synthesis method for a compound 2. Patent CN2018116183053 discloses a novel use of a compound 3 and its composition as agricultural fungicides.

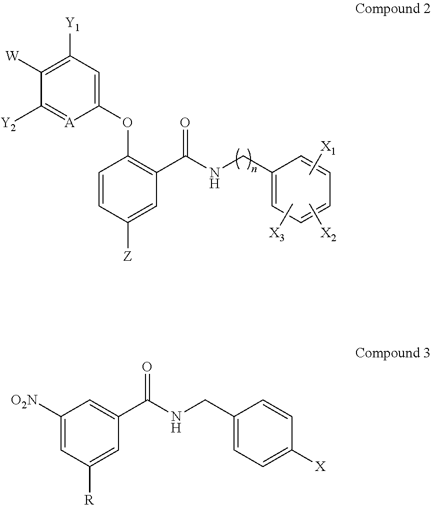

The inventor synthesized a series of N-benzylbenzamide-based compounds with specific structures, and found in conjunction with the bioassay data that the N-benzylbenzamide-based compound is a pigment synthesis inhibitor-based herbicide, and may be used for controlling weeds in field crop growing places or non-farming crop places. In view of the prior documents searched by the inventor, there has been no report on the use of an N-benzylbenzamide-based compound as a herbicide as disclosed in the present application.

### SUMMARY

Based on preliminary tests, the present application provides a novel use of an N-benzylbenzamide-based compound as a herbicidal active component, and a novel use of a herbicidal composition or a mixed formulation comprising the N-benzylbenzamide-based compound. Due to advantages of having a simple structure, stable properties, low toxicity, a short synthetic scheme and a low cost, the N-benzylbenzamide-based compound has better development and utilization values, and thereby has a great prospect in promotion and application.

The present application relates to use of an N-benzylbenzamide-based compound as a herbicide, wherein the herbicide comprises the N-benzylbenzamide-based compound of following general formula (I) or (II) or its salt as a component,

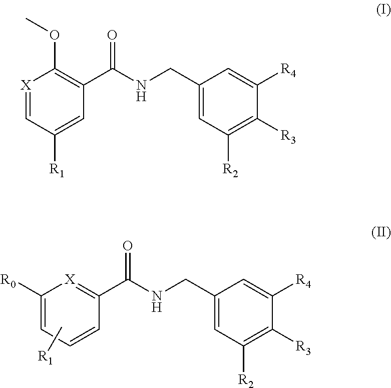

in the general formula (I) or (II),

R₀ is selected from methyl or methoxy;

R₁ is selected from hydrogen, C₁-C₄ alkyl, C₁-C₄ alkoxy, halo C₁-C₄ alkyl, halo C₁-C₄ alkoxy, halogen or cyano;

R₂, R₃ and R₄ are each independently selected from hydrogen, C₁-C₄ alkyl, C₁-C₄ alkoxy, halogen, cyano, halo C₁-C₄ alkyl, halo C₁-C₄ alkoxy or methylsulfonyl; and

X is selected from C or N.

More preferably, in the general formula (I) or (II),

R₀ is selected from methyl or methoxy;

R₁ is selected from hydrogen, methyl, methoxy, fluorine, chlorine, bromine or cyano;

R₂, R₃ and R₄ are each independently selected from hydrogen, methyl, methoxy, fluorine, chlorine, bromine, cyano and methylsulfonyl; and

X is selected from C or N.

The N-benzylbenzamide-based compound of general formula (I) or (II) of the present application may be prepared by a similar method with reference to the aforesaid documents.

The N-benzylbenzamide-based compound of general formula (I) may be prepared by a reaction Scheme I in a suitable solvent and a suitable alkaline condition with a synthetic scheme as follows:

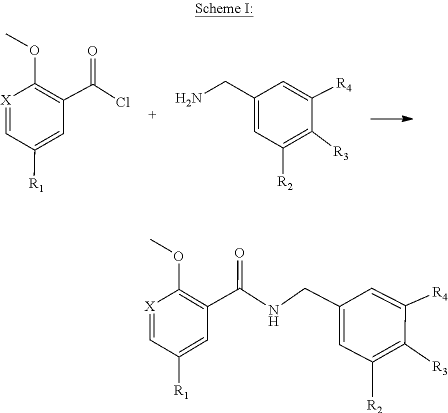

the reaction Scheme I is carried out in a suitable solvent, and the suitable solvent can be selected from tetrahydrofuran, acetonitrile, toluene, xylene, N,N-dimethylformamide, dichloromethane, chloroform, pyridine or the like; a suitable alkali can be selected from triethylamine, pyridine, potassium carbonate, sodium hydroxide or the like; a reaction temperature is between ice bath to a boiling temperature of the solvent, which is generally preferably 0 to 125° C.; and a reaction time is 30 minutes to 20 hours, which is generally preferably 1 to 6 hours.

Table 1 lists some of the compounds represented by the general formula (I). It shall be noted that the N-benzylbenzamide-based compound represented by the general formula (I) is not limited to the compounds listed in Table 1.

TABLE 1
Some of the N-benzylbenzamide-based compounds
represented by general formula (I)

| No. | R₁ | R₂ | R₃ | R₄ | X |
|---|---|---|---|---|---|
| A01 | —H | —F | —H | —H | C |
| A02 | —H | —H | —F | —H | C |
| A03 | —H | —F | —H | —F | C |
| A04 | —H | —F | —F | —H | C |
| A05 | —H | —CF₃ | —H | —H | C |
| A06 | —H | —H | —CF₃ | —H | C |
| A07 | —H | —Cl | —H | —F | C |
| A08 | —H | —Cl | —H | —Cl | C |
| A09 | —H | —Cl | —H | —H | C |
| A10 | —H | —Br | —H | —H | C |
| A11 | —H | —H | —Br | —H | C |
| A12 | —H | —Br | —H | —Br | C |
| A13 | —H | —Br | —Br | —H | C |
| A14 | —H | —CH₃ | —H | —H | C |
| A15 | —H | —H | —CH₃ | —H | C |
| A16 | —H | —CH₃ | —H | —CH₃ | C |
| A17 | —H | —CH₃ | —CH₃ | —H | C |
| A18 | —H | —OCH₃ | —H | —H | C |
| A19 | —H | —H | —OCH₃ | —H | C |
| A20 | —H | —OCH₃ | —H | —OCH₃ | C |
| A21 | —H | —OCH₃ | —OCH₃ | —H | C |
| A22 | —OCH₃ | —F | —H | —H | C |
| A23 | —OCH₃ | —H | —F | —H | C |
| A24 | —OCH₃ | —F | —H | —F | C |
| A25 | —OCH₃ | —F | —F | —H | C |
| A26 | —OCH₃ | —CF₃ | —H | —H | C |
| A27 | —OCH₃ | —H | —CF₃ | —H | C |
| A28 | —OCH₃ | —Cl | —H | —F | C |
| A29 | —OCH₃ | —Cl | —H | —Cl | C |
| A30 | —OCH₃ | —Cl | —Cl | —H | C |
| A31 | —OCH₃ | —Br | —H | —H | C |
| A32 | —OCH₃ | —H | —Br | —H | C |
| A33 | —OCH₃ | —Br | —H | —Br | C |
| A34 | —OCH₃ | —Br | —Br | —H | C |
| A35 | —OCH₃ | —CH₃ | —H | —H | C |
| A36 | —OCH₃ | —H | —CH₃ | —H | C |
| A37 | —OCH₃ | —CH₃ | —H | —CH₃ | C |
| A38 | —OCH₃ | —CH₃ | —CH₃ | —H | C |
| A39 | —OCH₃ | —OCH₃ | —H | —H | C |
| A40 | —OCH₃ | —H | —OCH₃ | —H | C |
| A41 | —OCH₃ | —OCH₃ | —H | —OCH₃ | C |
| A42 | —OCH₃ | —OCH₃ | —OCH₃ | —H | C |
| A43 | —F | —F | —H | —H | C |
| A44 | —F | —H | —F | —H | C |
| A45 | —F | —F | —H | —F | C |
| A46 | —F | —F | —F | —H | C |
| A47 | —F | —CF₃ | —H | —H | C |
| A48 | —F | —H | —CF₃ | —H | C |
| A49 | —F | —Cl | —H | —F | C |
| A50 | —F | —Cl | —H | —Cl | C |
| A51 | —F | —Cl | —Cl | —H | C |
| A52 | —F | —Br | —H | —H | C |
| A53 | —F | —H | —Br | —H | C |
| A54 | —F | —Br | —H | —Br | C |
| A55 | —F | —Br | —Br | —H | C |
| A56 | —F | —CH₃ | —H | —H | C |
| A57 | —F | —H | —CH₃ | —H | C |
| A58 | —F | —CH₃ | —H | —CH₃ | C |
| A59 | —F | —CH₃ | —CH₃ | —H | C |
| A60 | —F | —OCH₃ | —H | —H | C |
| A61 | —F | —H | —OCH₃ | —H | C |
| A62 | —F | —OCH₃ | —H | —OCH₃ | C |
| A63 | —F | —OCH₃ | —OCH₃ | —H | C |
| A64 | —CH₃ | —F | —H | —H | C |
| A65 | —CH₃ | —H | —F | —H | C |
| A66 | —CH₃ | —F | —H | —F | C |
| A67 | —CH₃ | —F | —F | —H | C |
| A68 | —CH₃ | —CF₃ | —H | —H | C |
| A69 | —CH₃ | —H | —CF₃ | —H | C |
| A70 | —CH₃ | —Cl | —H | —F | C |
| A71 | —CH₃ | —Cl | —H | —Cl | C |
| A72 | —CH₃ | —Cl | —Cl | —H | C |
| A73 | —CH₃ | —Br | —H | —H | C |
| A74 | —CH₃ | —H | —Br | —H | C |
| A75 | —CH₃ | —Br | —H | —Br | C |
| A76 | —CH₃ | —Br | —Br | —H | C |
| A77 | —CH₃ | —CH₃ | —H | —H | C |
| A78 | —CH₃ | —H | —CH₃ | —H | C |
| A79 | —CH₃ | —CH₃ | —H | —CH₃ | C |
| A80 | —CH₃ | —CH₃ | —CH₃ | —H | C |
| A81 | —CH₃ | —OCH₃ | —H | —H | C |
| A82 | —CH₃ | —H | —OCH₃ | —H | C |
| A83 | —CH₃ | —OCH₃ | —H | —OCH₃ | C |
| A84 | —CH₃ | —OCH₃ | —OCH₃ | —H | C |
| A85 | —H | —CH₃ | —H | —H | N |
| A86 | —H | —H | —CH₃ | —H | N |
| A87 | —H | —OCH₃ | —H | —H | N |
| A88 | —H | —H | —OCH₃ | —H | N |
| A89 | —H | —Cl | —H | —F | N |
| A90 | —H | —Cl | —F | —H | N |
| A91 | —H | —Cl | —H | —H | N |
| A92 | —H | —H | —H | —Cl | N |
| A93 | —H | —F | —H | —H | N |
| A94 | —H | —H | —F | —H | N |
| A95 | —H | —F | —H | —F | N |
| A96 | —H | —F | —F | —H | N |
| A97 | —H | —Br | —H | —H | N |
| A98 | —H | —Br | —H | —F | N |
| A99 | —H | —Br | —H | —Br | N |

The N-benzylbenzamide-based compound of general formula (II) may be prepared by a reaction Scheme II in a suitable solvent and a suitable alkaline condition with a synthetic scheme as follows:

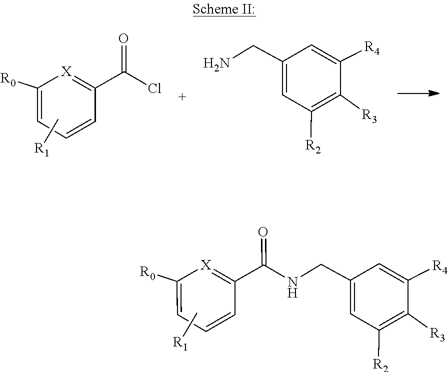

the reaction Scheme II is carried out in a suitable solvent, and the suitable solvent can be selected from tetrahydrofuran, acetonitrile, toluene, xylene, N,N-dimethylformamide, dichloromethane, chloroform, pyridine or the like; a suitable alkali is selected from triethylamine, pyridine, potassium carbonate, sodium hydroxide or the like; a reaction temperature is between ice bath to a boiling temperature of the solvent, which is generally preferably 0 to 125° C.; and a reaction time is 30 minutes to 20 hours, which is generally preferably 1 to 6 hours.

Table 2 lists some of the compounds represented by the general formula (II). It shall be noted that the N-benzylbenzamide-based compound represented by the general formula (II) is not limited to the compounds listed in Table 2.

TABLE 2
Some of the N-benzylbenzamide-based compounds
represented by general formula (II)

| No. | R₀ | R₁ | R₂ | R₃ | R₄ | X |
|---|---|---|---|---|---|---|
| B01 | —CH₃ | —H | —F | —H | —H | C |
| B02 | —CH₃ | —H | —H | —F | —H | C |
| B03 | —CH₃ | —H | —F | —H | —F | C |
| B04 | —CH₃ | —H | —F | —F | —H | C |
| B05 | —CH₃ | —H | —CF₃ | —H | —H | C |
| B06 | —CH₃ | —H | —H | —CF₃ | —H | C |
| B07 | —CH₃ | —H | —Cl | —H | —F | C |
| B08 | —CH₃ | —H | —Cl | —H | —Cl | C |
| B09 | —CH₃ | —H | —Cl | —Cl | —H | C |
| B10 | —CH₃ | —H | —Br | —H | —H | C |
| B11 | —CH₃ | —H | —H | —Br | —H | C |
| B12 | —CH₃ | —H | —Br | —H | —Br | C |
| B13 | —CH₃ | —H | —Br | —Br | —H | C |
| B14 | —CH₃ | —H | —CH₃ | —H | —H | C |
| B15 | —CH₃ | —H | —H | —CH₃ | —H | C |
| B16 | —CH₃ | —H | —CH₃ | —H | —CH₃ | C |
| B17 | —CH₃ | —H | —F | —H | —CH₃ | C |
| B18 | —CH₃ | —H | —OCH₃ | —H | —H | C |
| B19 | —CH₃ | —H | —H | —OCH₃ | —H | C |
| B20 | —CH₃ | —H | —OCH₃ | —H | —OCH₃ | C |
| B21 | —CH₃ | —H | —OCH₃ | —OCH₃ | —H | C |
| B22 | —CH₃ | —H | —F | —H | —H | N |
| B23 | —CH₃ | —H | —H | —F | —H | N |
| B24 | —CH₃ | —H | —F | —H | —F | N |
| B25 | —CH₃ | —H | —F | —F | —H | N |
| B26 | —CH₃ | —H | —H | —H | —H | N |
| B27 | —CH₃ | —H | —F | —H | —Cl | N |
| B28 | —CH₃ | —H | —F | —Cl | —H | N |
| B29 | —CH₃ | —H | —Cl | —H | —Cl | N |
| B30 | —CH₃ | —H | —Cl | —F | —H | N |
| B31 | —CH₃ | —H | —Cl | —H | —H | N |
| B32 | —CH₃ | —H | —H | —Br | —H | N |
| B33 | —CH₃ | —H | —Br | —H | —Br | N |
| B34 | —CH₃ | —H | —Br | —Br | —H | N |
| B35 | —CH₃ | —H | —CH₃ | —H | —H | N |
| B36 | —CH₃ | —H | —H | —CH₃ | —H | N |
| B37 | —CH₃ | —H | —OCH₃ | —H | —H | N |
| B38 | —CH₃ | —H | —H | —OCH₃ | —H | N |
| B39 | —OCH₃ | —H | —OCH₃ | —H | —H | N |
| B40 | —OCH₃ | —H | —H | —OCH₃ | —H | N |
| B41 | —OCH₃ | —H | —F | —H | —H | N |
| B42 | —OCH₃ | —H | —H | —F | —H | N |
| B43 | —OCH₃ | —H | —F | —H | —F | N |
| B44 | —OCH₃ | —H | —F | —F | —H | N |
| B45 | —OCH₃ | —H | —H | —H | —H | N |
| B46 | —OCH₃ | —H | —F | —H | —Cl | N |
| B47 | —OCH₃ | —H | —F | —Cl | —H | N |
| B48 | —OCH₃ | —H | —Cl | —H | —Cl | N |
| B49 | —OCH₃ | —H | —Cl | —H | —H | N |
| B50 | —OCH₃ | —H | —Br | —H | —H | N |
| B51 | —OCH₃ | —H | —H | —Br | —H | N |
| B52 | —OCH₃ | —H | —Br | —H | —Br | N |
| B53 | —OCH₃ | —H | —Br | —Br | —H | N |
| B54 | —OCH₃ | —H | —CH₃ | —H | —H | N |
| B55 | —OCH₃ | —H | —H | —CH₃ | —H | N |
| B56 | —OCH₃ | —H | —OCH₃ | —H | —H | N |
| B57 | —OCH₃ | —H | —H | —OCH₃ | —H | N |
| B58 | —OCH₃ | —H | —OCH₃ | —H | —H | N |
| B59 | —OCH₃ | —H | —H | —OCH₃ | —H | N |

The weeds treated with the N-benzylbenzamide-based compound according to the present disclosure present a typical “albino effect”, which means that the symptoms of the weeds after the treatment with the compound are leaf albino, chlorosis, and death. The weed symptoms caused by the N-benzylbenzamide-based compound are similar to those caused by the herbicides acting on the carotenoid biosynthetic route such as mesotrione (HPPD inhibitor), diflufenican (PDS inhibitor), but are different from those caused by flumioxazin (PPO inhibitor) which interferes with chlorophyll biosynthesis. Thus, it may be speculated that the N-benzylbenzamide-based compound according to the present disclosure may act on the carotenoid biosynthetic route and belongs to a pigment synthesis inhibitor. It can be learn from the tests that the N-benzylbenzamide-based compound has a higher herbicidal activity than the existing commercial pigment synthesis inhibitor-based herbicides and thereby may be used for controlling weeds that are resistant to existing herbicides, especially for controlling weeds that are resistant to ALS inhibitor-based, PSII inhibitor-based, ACCase inhibitor-based and EPSP inhibitor-based herbicides.

In the present disclosure, the weeds shall be explained in a broadest or broader meaning, comprising all types of plant species that grow in undesirable places. In the present disclosure, the “undesirable places” are divided into field crop growing places and non-farming crop places. The field crop growing places refer to the farms or the fields in the common sense. The non-farming crop places refer to places other than the field crop growing places where the weeds are not expected to grow, which may comprise vineyards, apple orchards, citrus orchards, banana farms, coffee plantations, tea plantations, rubber plantations, oil palm plantations, coconut plantations and the like.

The N-benzylbenzamide-based compound and the herbicide comprising the N-benzylbenzamide-based compound as a component may be used to control the following weeds growing in the following crop fields.

Crop genera in the Dicotyledoneae comprises: Gossypium, Glycine, Beta, Phaseolus, Pisum, Solanum, Linum, Lycopersicon, Arachis, Brassica, Lactuca, Cucumis, Cucurbita, and the like.

Crop genera in the Monocotyledoneae comprises: Oryza, Zea, Triticum, Hordeum, Avena, Panicum, Saccharum, Bromelia, Asparagus, Allium, and the like.

Weed genera in the dicotyledoneae comprises: Sinapis, Brassica, Galium, Stellaria, Chenopodium, Kochia, Urtica, Senecio, Amaranthus, Portulaca, Xanthium, Ipomoea, Polygonum, Ambrosia, Cirsium, Sonchus, Solanum, Veronica, Datura, Viola, Papaver, Centaurea, Galinsoga, Rotala, Lindernia, Sesbania, Trifolium, Abutilon, Lamium, Matricaria, Artemisia, Pharbitis, and the like.

Weed genera in the monocotyledoneae comprises Echinochloa, Setaria, Panicum, Digitaria, Phleum, Poa, Festuca, Lolium, Bromus, Avena, Cyperus, Sorghum, Agropyron, Monochoria, Fimbristylis, Sagittaria, Scirpus, Paspalum, Ischaemum, Agrostis, Alopecurus, Cynodon, Commelina, Brachiaria, Leptochloa, or the like.

Furthermore, the N-benzylbenzamide-based compound and the herbicide comprising the N-benzylbenzamide-based compound as a component may be used to control the following representative farmland weeds. The farmland weeds comprise:

barnyard grass, pondweed, Monochoria vaginalis, Cyperus difformis, Scirpus planiculmis, Leptochloa chinensis, Avena fatua, Digitaria sanguinalis, goosegrass, green bristlegrass, Alopecurus aequalis, flixweed, Capsella bursa-pastoris, Lepidium apetalum, Humulus scandens, Polygonum aviculare, Convolvulus arvensis, Fallopia convolvulus, Cyperus rotundus, Chenopodium album, Polygonum lapathifolium, Amaranthus retroflexus, Portulaca oleracea, Amaranthus blitum, Cuscuta chinensis, piemarker, Galium spurium, Eclipta prostrata, Xanthium strumarium, bermudagrass, cogon, Solanum nigrum, Myosoton aquaticum, Chromolaena odorata, Ageratum conyzoides, Rotala rotundifolia, Artemisia argyi, dandelion, Erigeron annuus, Hemisteptia lyrata, Artemisia capillaris, Ixeris polycephala, field thistle, setose thistle, Elsholtzia ciliata, woundwort, Acalypha australis, or the like.

Furthermore, the present application provides a use of a composition as an agricultural herbicide, wherein a herbicidal component of the composition is the N-benzylbenzamide-based compound or its salt, and the composition further comprises an agriculturally acceptable carrier. Regarding the agricultural application method of the composition, the inventor has learned from tests that a good weed control effect can be obtained by applying the composition to the soil in a pre-emergence soil treatment manner or to the weeds in a post-emergence stem and leaf spraying manner.

In order to obtain an ideal weed control effect, the dosage of the N-benzylbenzamide-based compound or its salt varies depending on various factors, such as the compounds used, the crops to be protected, the weeds to be controlled, the growth degree of the weeds, climatic conditions, application methods, and the dosage forms adopted. The inventor verified the dosage of the composition and found that the dosage of the N-benzylbenzamide-based compound or its salt per hectare is 10 g to 5000 g. At this dosage, the composition may exhibit an ideal weed control effect. The N-benzylbenzamide-based compound or its salt has good herbicidal activity both pre- and post-emergence, and the symptoms are leaf albino, wilting and death. In addition, a good weed control effect can also be achieved at a very low dosage under a suitable condition of temperature and light. Further preferably, it is generally more suitable that the dosage of the N-benzylbenzamide-based compound or its salt is 10 g to 1000 g per hectare. In the composition, the weight percentage of the N-benzylbenzamide-based compound or its salt is advised to be between 0.1% and 99.0%. The inventor has discovered from the research that the N-benzylbenzamide-based compound or its salt, which is controlled at an appropriate concentration, may serve as a selective herbicide.

Furthermore, the present application provides use of a mixed formulation as an agricultural herbicide. The mixed formulation comprises the N-benzylbenzamide-based compound or its salt as a herbicidal active component, and further comprises a herbicidal component serving as an acetyl-CoA carboxylase inhibitor, an acetolactate synthase inhibitor, an auxin herbicide, a carotenoid biosynthesis inhibitor, a photosynthesis inhibitor, or a cell wall biosynthesis inhibitor, and the herbicidal component may exhibit a compound synergistic effect when being used in conjunction with the N-benzylbenzamide-based compound or its salt.

Specifically, the N-benzylbenzamide-based compound or its salt according to the present disclosure may have a significant synergistic effect when used as a herbicidal composition with herbicides acting on the photosynthetic system II (PSII) (such as triazine-based, pyridazinone-based, benzothiadiazinone-based, phenylpyridazine-based, and substituted urea-based herbicides), and it also has a good controlling effect on older weeds, and further, the action time is significantly accelerated, and the weeds exhibit obvious symptoms of albinism on the day of application.

More specifically, the weight ratio of the N-benzylbenzamide-based compound or its salt according to the present disclosure to the PSII herbicide in the composition is 1:20 to 20:1; the weight percentage of the N-benzylbenzamide-based compound or its salt and the PSII herbicide in the formulation is advised to be between 0.1% and 99.0%; and the appropriate dosage is 10 g to 1000 g of the active ingredients per hectare.

More specifically, the weight ratio of the N-benzylbenzamide-based compound or its salt according to the present disclosure to atrazine in the composition is 1:10 to 10:1; the weight percentage of the N-benzylbenzamide-based compound or its salt and the PSII herbicide in the formulation is advised to be between 0.1% and 99.0%; and the appropriate dosage is 10 g to 1000 g of the active ingredients per hectare. The composition can significantly reduce the amount of atrazine, which thereby reduces the phytotoxicity of atrazine residues on succeeding crops, and significantly improves the controlling effect on older gramineous weeds. In addition, the action time is significantly accelerated.

Those skilled in the art should know that in the use of the composition provided by the present application as an agricultural herbicide, and in the use of the mixed formulation as an agricultural herbicide, the composition or the mixed formulation is generally used in agriculture in the form of a formulation which further comprises an agriculturally acceptable carrier. The N-benzylbenzamide-based compound or its salt, or other herbicidal active components compounded with the N-benzylbenzamide-based compound may serve as an active component to be dissolved or dispersed in a carrier or formulated into a formulation so as to be easily dispersed when being used as a herbicide. These chemical formulations may be made into, but not limited to, any one of the dosage forms of wettable powders, water-dispersible granules, emulsion in water, oil dispersions, emulsifiable concentrates or suspensions. For those skilled in the art of formulation processing, one or more suitable carriers or adjuvants may be selected.

Taking the water-dispersible granules as an example, the corresponding carriers or adjuvants used to implement the present invention are well-known to those skilled in the art. The dispersant is for example one or more of polycarboxylate (such as TERSPERSE 2700, T36, GY-D06, etc.), lignosulfonate, and alkyl naphthalene sulfonate; the wetting agent is for example one or more of alkyl sulfate, alkyl sulfonate, and naphthalene sulfonate; the disintegrant is for example one or more of ammonium sulfate, urea, sucrose, and glucose; the binder is for example one or more of diatomaceous earth, corn starch, polyvinyl alcohol (PVA), and carboxymethyl(ethyl) cellulose; the safener is MON13900 (product of Monsanto); and the filler is for example one or more of diatomite, kaolin, white carbon black, light calcium, talc, attapulgite, and syderolife.

Taking the wettable powders as an example, the corresponding carriers or adjuvants used to implement the present invention are well-known to those skilled in the art. The usable dispersant is for example one or more of polycarboxylates (TERSPERSE 2700, T36, GY-D06, etc.), lignosulfonates (Ufoxane 3A, Borresperse NA, Borresperse CA-SA, etc.), naphthalene and alkyl naphthalene formaldehyde condensate sulfonates (NNO, MF, Morwet D-425, Tamol NN, TERSPERSE2020, etc.), nekal BX (sodium dibutyl naphthalene sulfonate), EO-PO block polyethers, alkylphenol polyoxyethylene ether phosphate, and alkylphenol polyoxyethylene ether formaldehyde condensate sulfate (SOPA); the wetting agent is for example one or more of sulfates (K-12), sulfonates (ABS-Na, BX, Terwet 1004, etc.), and composite wetting agents (Morwet EFW); and the filler is for example one or more of diatomaceous earth, kaolin, light calcium, talc, white carbon black, attapulgite, syderolife, ammonium sulfate, urea, sucrose, glucose, corn starch, sodium sulfate, and sodium polyphosphate.

Taking the oil dispersions as an example, the corresponding carriers or adjuvants used to implement the present invention are well-known to those skilled in the art. The usable dispersant is for example one or more of polycarboxylate, lignosulfonate, alkyl naphthalene sulfonate (diffusant NNO), and TERSPERSE 2020 (alkyl naphthalene sulfonates); the emulsifier is for example one or more of Agricultural Emulsifier 700# (with a common name of alkylphenol formaldehyde resin polyoxyethylene ether), Agricultural Emulsifier 2201, Span-60# (with a common name of sorbitan stearate), Tween-60# (with a common name of polyoxyethylene sorbitan stearate), Agricultural Emulsifier 1601# (with a common name of triphenylethylphenol polyoxypropylene polyoxyethylene block polymer), and TERSPERSE 4894; the wetting agent is for example one or more of alkylphenol polyoxyethylene ether formaldehyde condensate sulfate, alkylphenol polyoxyethylene ether phosphate, phenethylphenol polyoxyethylene ether phosphate, alkyl sulfate, alkyl sulfonate, naphthalene sulfonate, and TERSPERSE 2500; the thickener is for example one or more of xanthan gum, polyvinyl alcohol, bentonite, and magnesium aluminum silicate; the preservative is for example one or more of formaldehyde, benzoic acid, and sodium benzoate; the safener is MON13900; the antifoaming agent is for example silicone antifoaming agent; the antifreezing agent is for example one or more of ethylene glycol, propylene glycol, glycerin, urea, and inorganic salts such as sodium chloride; and the water is deionized water.

Taking the suspensions as an example, the dispersant selected and used is for example one or more of polycarboxylate, lignin sulfonate, alkyl naphthalene sulfonate (diffusant NNO), and TERSPERSE 2020 (alkyl naphthalene sulfonate); the emulsifier is for example one or more of BY (castor oil polyoxyethylene ether) series emulsifiers (BY-110, BY-125, and BY-140), Agricultural Emulsifier 700# (with a common name of alkylphenol formaldehyde resin polyoxyethylene ether), Agricultural Emulsifier 2201, Span-60# (with a common name of sorbitan monostearate), Tween-60# (with a common name of sorbitan monostearate polyoxyethylene ether), Agricultural Emulsifier 1601# (with a common name of phenethyl phenol polyoxyethylene polyoxypropylene ether), and TERSPERSE 4894; the wetting agent is for example one or more of alkylphenol polyoxyethylene ether formaldehyde condensate sulfate, alkylphenol polyoxyethylene ether phosphate, phenethylphenol polyoxyethylene ether phosphate, alkyl sulfate, alkyl sulfonate, naphthalene sulfonate, and TERSPERSE 2500 (produced by Huntsman, USA); the thickener is for example one or more of white carbon black, polyvinyl alcohol, bentonite, and magnesium aluminum silicate; the safener is MON13900; the antifreezing agent is for example one or more of ethylene glycol, propylene glycol, glycerin, urea, and inorganic salts such as sodium chloride; the dispersion medium is for example one or more of soybean oil, rapeseed oil, cottonseed oil, corn oil, castor oil, palm oil, epoxidized soybean oil, methyl oleate and its methylated oils, diesel, engine oil, and mineral oil; and the ester solvent is for example one or more of dimethyl phthalate, dibutyl phthalate, ethyl acetate, and methyl benzoate.

The present disclosure has following advantageous effect and advantages.

The present disclosure provides a novel use of the N-benzylbenzamide-based compound or its salt in weed control, which expands the application of the N-benzylbenzamide-based compound in the agricultural field. The N-benzylbenzamide-based compound or its salt serves as a herbicide for controlling weeds in field crop growing places or non-farming crop places, has a high herbicidal activity and may further be used to control weeds that are resistant to existing herbicides, especially suitable for controlling weeds that are resistant to ALS inhibitor-based, PSII inhibitor-based, ACCase inhibitor-based and EPSP inhibitor-based herbicides.

The present application further specifies that a composition comprising the N-benzylbenzamide-based compound or its salt and a PSII inhibitor-based herbicide still exhibits a good controlling effect on weeds in the field crop growing places or the non-farming crop places. This fully shows that the N-benzylbenzamide-based compound or its salt has great development potential, and provides a new idea for the development of new green agricultural chemicals.

The composition or the mixed formulation comprising the N-benzylbenzamide-based compound or its salt according to the present disclosure may be made into various dosage forms that satisfy the needs of agricultural production, such as suspensions, water-dispersible granules, wettable powders, oil dispersions, or the like, which are safe to the crops and meet the safety requirements for agricultural chemical formulations. The present disclosure enriches the variety of existing herbicides and can bring considerable market benefits.

### DETAILED DESCRIPTION

For easy understanding of the purpose, technical solutions and effects of the present disclosure, the present disclosure will now be further described in detail in conjunction with examples.

(I). Synthesis of N-Benzylbenzamide-Based Compound

### Example 1: Preparation of N-(3-fluorobenzylamine)-2-methoxybenzamide (Compound A01)

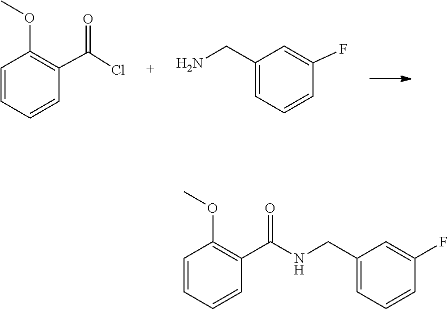 [SMILES: COc1ccccc1C(=O)Cl ; NCc1cccc(F)c1 ; COc1ccccc1C(=O)NCc1cccc(F)c1]

125 g (1.0 mol) of 3-fluorobenzylamine and 150 g (1.5 mol) of triethylamine were dissolved in 1.5 L of toluene, and stirred for 30 minutes in ice bath, and then 170 g (1.0 mol) of 2-methoxybenzoyl chloride dissolved in 500 ml of toluene was added dropwise before being stirred at room temperature for 1.5 hours; the mixture was washed by adding 200 ml of saturated sodium bicarbonate solution and then washed with 200 ml of deionized water and 200 ml of saturated brine in sequence, dried with anhydrous sodium sulfate, and then subjected to reduced pressure to remove the solvent, and the residue was subjected to silica gel column chromatography to obtain 230 g of product with a yield of 88.8%. ¹H NMR (500 MHz, CDCl₃) δ 8.24 (d, J=8.0 Hz, 2H), 7.45 (t, J=8.0 Hz, 1H), 7.27-7.31 (m, 1H), 7.03-7.14 (m, 3H), 6.90-6.99 (m, 2H), 4.68 (d, J=5.0 Hz, 2H), 3.93 (s, 3H).

### Example 2: Preparation of N-(4-fluorobenzyl)-2-hydroxyaniline (Compound A02)

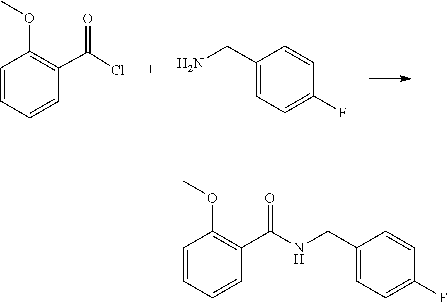 [SMILES: COc1ccccc1C(=O)Cl ; NCc1ccc(F)cc1 ; COc1ccccc1C(=O)NCc1ccc(F)cc1]

125 g (1.0 mol) of 4-fluorobenzylamine and 150 g (1.5 mol) of triethylamine were dissolved in 1.5 L of toluene, and stirred for 30 minutes in ice bath, and then 170 g (1.0 mol) of 2-methoxybenzoyl chloride dissolved in 500 ml of toluene was added dropwise before being stirred at room temperature for 1.5 hours; the mixture was washed by adding 200 ml of saturated sodium bicarbonate solution and then washed with 200 ml of deionized water and 200 ml of saturated brine in sequence, dried with anhydrous sodium sulfate, and then subjected to reduced pressure to remove the solvent, and the residue was subjected to silica gel column chromatography to obtain 220 g of product with a yield of 84.9%. ¹H NMR (500 MHz, CDCl₃) δ 8.23-8.25 (m, 1H), 8.18 (br, 1H), 7.44-7.47 (m, 1H), 7.32-7.35 (m, 2H), 6.97-7.11 (m, 4H), 4.59 (d, J=5.5 Hz, 2H), 3.92 (s, 3H).

### Example 3: Preparation of N-(3,5 difluorobenzyl)-2-methoxybenzamide (Compound A03)

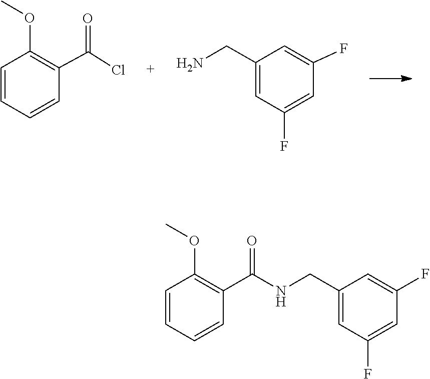 [SMILES: COc1ccccc1C(=O)Cl ; NCc1cc(F)cc(F)c1 ; COc1ccccc1C(=O)NCc1cc(F)cc(F)c1]

143 g (1.0 mol) of 3,5-fluorobenzylamine and 150 g (1.5 mol) of triethylamine were dissolved in 1.5 L of toluene, and stirred for 30 minutes in ice bath, and then 170 g (1.0 mol) of 2-methoxybenzoyl chloride dissolved in 500 ml of toluene was added dropwise before being stirred at room temperature for 1.5 hours; the mixture was washed by adding 200 ml of saturated sodium bicarbonate solution and then washed with 200 ml of deionized water and 200 ml of saturated brine in sequence, dried with anhydrous sodium sulfate, and then subjected to reduced pressure to remove the solvent, and the residue was subjected to silica gel column chromatography to obtain 245 g of product with a yield of 88.4%. ¹H NMR (500 MHz, CDCl₃) δ 8.18-8.34 (m, 2H), 7.46-7.52 (m, 1H), 7.08-7.14 (m, 1H), 7.01 (d, J=8.3 Hz, 1H), 6.88 (d, J=6.1 Hz, 2H), 6.67-6.75 (m, 1H), 4.67 (d, J=6.0 Hz, 2H), 3.97 (s, 3H).

### Example 4: Preparation of N-(3-chloro-5-fluorobenzyl)-2-methoxybenzamide (Compound A07)

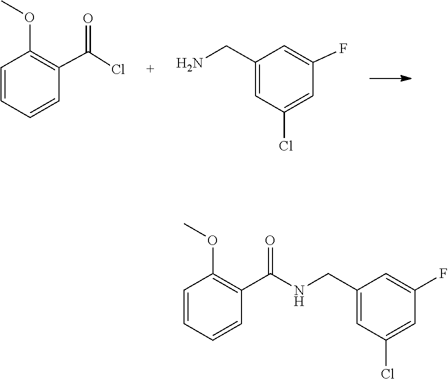 [SMILES: COc1ccccc1C(=O)Cl ; NCc1cc(F)cc(Cl)c1 ; COc1ccccc1C(=O)NCc1cc(F)cc(Cl)c1]

159 g (1.0 mol) of 3-chloro-5-fluorobenzylamine and 150 g (1.5 mol) of triethylamine were dissolved in 1.5 L of toluene, and stirred for 30 minutes in ice bath, and then 170 g (1.0 mol) of 2-methoxybenzoyl chloride dissolved in 500 ml of toluene was added dropwise before being stirred at room temperature for 1.5 hours; the mixture was washed by adding 200 ml of saturated sodium bicarbonate solution and then washed with 200 ml of deionized water and 200 ml of saturated brine in sequence, dried with anhydrous sodium sulfate, and then subjected to reduced pressure to remove the solvent, and the residue was subjected to silica gel column chromatography to obtain 260 g of product with a yield of 88.7%. ¹H NMR (500 MHz, CDCl₃) δ 8.19-8.33 (m, 2H), 7.48 (t, J=6.9 Hz, 1H), 7.07-7.16 (m, 1H), 7.00 (d, J=8.3 Hz, 1H), 6.88 (d, J=6.1 Hz, 2H), 6.69 (t, J=8.9 Hz, 1H), 4.66 (d, J=6.0 Hz, 2H), 3.97 (s, 3H).

### Example 5: Preparation of N-(3-chlorobenzyl)-2-methoxybenzamide (Compound A09)

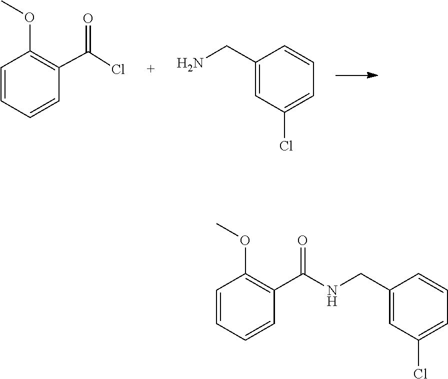 [SMILES: COc1ccccc1C(=O)Cl ; NCc1cccc(Cl)c1 ; COc1ccccc1C(=O)NCc1cccc(Cl)c1]

141 g (1.0 mol) of 3-chlorobenzylamine and 150 g (1.5 mol) of triethylamine were dissolved in 1.5 L of toluene, and stirred for 30 minutes in ice bath, and then 170 g (1.0 mol) of 2-methoxybenzoyl chloride dissolved in 500 ml of toluene was added dropwise before being stirred at room temperature for 1.5 hours; the mixture was washed by adding 200 ml of saturated sodium bicarbonate solution and then washed with 200 ml of deionized water and 200 ml of saturated brine in sequence, dried with anhydrous sodium sulfate, and then subjected to reduced pressure to remove the solvent, and the residue was subjected to silica gel column chromatography to obtain 260 g of product with a yield of 94.5%. ¹H NMR (500 MHz, CDCl₃) δ 8.00-8.28 (m, 2H), 7.45-7.51 (m, 1H), 7.35 (t, J=1.7 Hz, 1H), 7.22-7.31 (m, 3H), 7.05-7.14 (m, 1H), 6.99 (d, J=8.3 Hz, 1H), 4.67 (d, J=5.8 Hz, 2H), 3.95 (s, 3H).

### Example 6: Preparation of 2-methoxy-N-(3-methylbenzyl)benzamide (Compound A14)

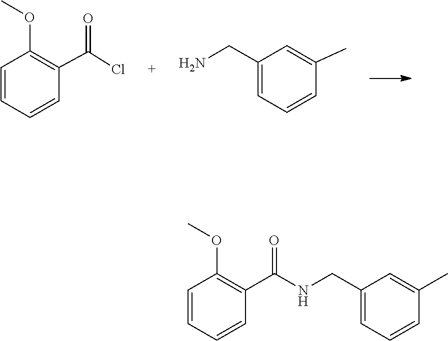

121 g (1.0 mol) of 3-methylbenzylamine and 150 g (1.5 mol) of triethylamine were dissolved in 1.5 L of toluene, and stirred for 30 minutes in ice bath, and then 170 g (1.0 mol) of 2-methoxybenzoyl chloride dissolved in 500 ml of toluene was added dropwise before being stirred at room temperature for 1.5 hours; the mixture was washed by adding 200 ml of saturated sodium bicarbonate solution and then washed with 200 ml of deionized water and 200 ml of saturated brine in sequence, dried with anhydrous sodium sulfate, and then subjected to reduced pressure to remove the solvent, and the residue was subjected to silica gel column chromatography to obtain 210 g of product with a yield of 82.4%. ¹H NMR (500 MHz, CDCl₃) δ 8.26 (dd, J=7.8, 1.9 Hz, 1H), 8.18 (br, 1H), 7.44-7.48 (m, 1H), 7.20-7.28 (m, 1H), 7.14-7.20 (m, 2H), 7.06-7.13 (m, 2H), 6.97 (d, J=8.3 Hz, 1H), 4.66 (d, J=5.7 Hz, 2H), 3.92 (s, 3H), 2.35 (s, 3H).

### Example 7: Preparation of N-(3-fluorobenzyl)-2,5-dimethoxybenzamide (Compound A22)

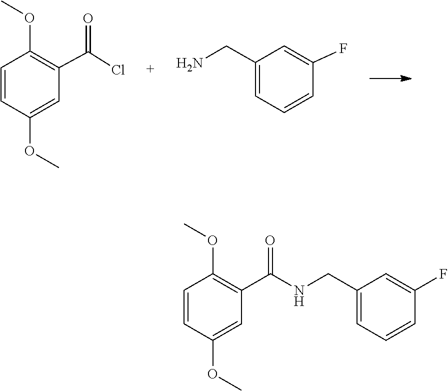 [SMILES: COc1ccc(OC)c(C(=O)Cl)c1 ; NCc1cccc(F)c1 ; COc1ccc(OC)c(C(=O)NCc2cccc(F)c2)c1]

125 g (1.0 mol) of 3-fluorobenzylamine and 150 g (1.5 mol) of triethylamine were dissolved in 1.5 L of toluene, and stirred for 30 minutes in ice bath, and then 200 g (1.0 mol) of 2,5-dimethoxybenzoyl chloride dissolved in 500 ml of toluene was added dropwise before being stirred at room temperature for 1.5 hours; the mixture was washed by adding 200 ml of saturated sodium bicarbonate solution and then washed with 200 ml of deionized water and 200 ml of saturated brine in sequence, dried with anhydrous sodium sulfate, and then subjected to reduced pressure to remove the solvent, and the residue was subjected to silica gel column chromatography to obtain 260 g of product with a yield of 90.0%. ¹H NMR (500 MHz, CDCl₃) δ 8.37 (s, 1H), 7.79 (d, J=3.3 Hz, 1H), 7.32-7.27 (m, 1H), 7.12 (d, J=7.7 Hz, 1H), 7.09-7.03 (m, 1H), 7.00 (dd, J=8.9, 3.3 Hz, 1H), 6.97-6.87 (m, 2H), 4.66 (d, J=5.8 Hz, 2H), 3.88 (d, J=2.7 Hz, 3H), 3.80 (d, J=2.9 Hz, 3H).

### Example 8: Preparation of 3-methyl-N-(3-methylbenzyl)benzamide (Compound B14)

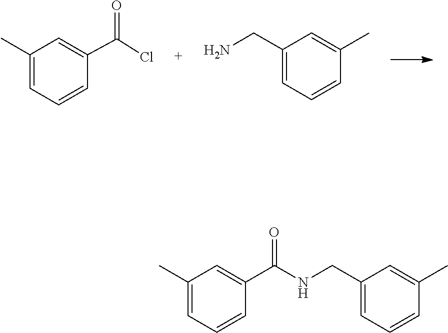

121 g (1.0 mol) of 3-methylbenzylamine and 150 g (1.5 mol) of triethylamine were dissolved in 1.5 L of toluene, and stirred for 30 minutes in ice bath, and then 154 g (1.0 mol) of 3-methylbenzoyl chloride dissolved in 500 ml of toluene was added dropwise before being stirred at room temperature for 1.5 hours; the mixture was washed by adding 200 ml of saturated sodium bicarbonate solution and then washed with 200 ml of deionized water and 200 ml of saturated brine in sequence, dried with anhydrous sodium sulfate, and then subjected to reduced pressure to remove the solvent, and the residue was subjected to silica gel column chromatography to obtain 212 g of product with a yield of 88.7%. ¹H NMR (500 MHz, CDCl₃) δ 7.62 (s, 1H), 7.59-7.54 (m, 1H), 7.31 (d, J=4.7 Hz, 2H), 7.24 (d, J=7.6 Hz, 1H), 7.19-7.14 (m, 2H), 7.12 (d, J=7.6 Hz, 1H), 6.36 (s, 1H), 4.61 (d, J=5.6 Hz, 2H), 2.39 (s, 3H), 2.35 (s, 3H)

(II). Preparation of Herbicide or Herbicidal Composition Comprising the N-Benzylbenzamide-Based Compound

### Example 9: Wettable Powders with 10% of the N-Benzylbenzamide-Based Compound (Compound B14)

Components and proportions thereof in the wettable powders:

| | |
|---|---|
| Compound B14 | 10% |
| Sodium lauryl sulfate | 3% |
| Sodium Lignosulfonate | 3% |
| Naphthalenesulfonic acid formaldehyde condensate | 5% |
| Light calcium carbonate | balance. |

The added amount of each component was in the percentage by weight, and the active compounds were added in an amount on 100% basis. The compound B14 and other components were thoroughly mixed and pulverized by a jet mill to obtain a wettable powder product comprising 10% of the N-benzylbenzamide-based compound (compound B14).

### Example 10: Water Emulsion with 10% of the N-Benzylbenzamide-Based Compound (Compound A09)

Components and proportions thereof in the water emulsion:

| | |
|---|---|
| Compound A09 | 10% |
| Ethylene glycol | 5% |
| Nonylphenol polyglycol ether | 3% |
| Sodium Lignosulfonate | 5% |
| Carboxymethyl cellulose | 1% |
| 75% silicone oil water emulsion | 0.4% |
| Water | balance. |

The added amount of each component was in the percentage by weight, and the active compounds were added in an amount on 100% basis. The compound A09 and other components were mixed thoroughly in a stirred tank to obtain a water emulsion product comprising 10% of the N-benzylbenzamide-based compound (compound A09).

### Example 11: Oil Dispersion with 10% of the N-Benzylbenzamide-Based Compound (Compound A85)

Components and proportions thereof in the oil dispersion:

| | |
|---|---|
| Compound A85 | 10% |
| Rhodia V0/02N | 12% |
| Organobentonite | 3% |
| White carbon black | 3% |
| Citric acid | 2% |
| Methyl oleate | balance |

The added amount of each component was in the percentage by weight, and the active compounds were added in an amount on 100% basis. The compound A85 and other components were mixed thoroughly in a stirred tank to obtain an oil dispersion product comprising 10% of the N-benzylbenzamide-based compound (compound A85).

### Example 12: Emulsifiable Concentrate with 10% of the N-Benzylbenzamide-Based Compound (Compound B07)

Components and proportions thereof in the emulsifiable concentrate:

| | |
|---|---|
| Compound B07 | 10% |
| Calcium dodecylbenzene sulfonate | 4% |
| Agricultural Emulsifier 600# | 10% |
| Methyl oleate | balance |

The added amount of each component was in the percentage by weight, and the active compounds were added in an amount on 100% basis. The compound B07 and other components were mixed thoroughly in a stirred tank to obtain an emulsifiable concentrate product comprising 10% of the N-benzylbenzamide-based compound (compound B07).

Several herbicides comprising the N-benzylbenzamide-based compound provided by Examples 9-12 were merely an illustration for the preparation of the herbicide or the herbicidal composition formulation comprising the N-benzylbenzamide-based compound.

(III). Efficacy Test of the Herbicide or Herbicidal Composition Comprising the N-Benzylbenzamide-Based Compound

### Example 13: Synergistic Test of Compound A02 with Atrazine

Seeds of green bristlegrass were sown in plastic pots with a diameter of 9 cm, and each pot was sown with 20 seeds that were covered with 0.5 cm of soil and cultivated in a greenhouse at 25-35° C. under natural light and natural humidity. When the green bristlegrass was in the stage where 3 to 4 leaves were grown, applying the agent with an automatic spraying system. 29 treatments were provided in total, in which there were four single-agent treatments with a single agent of A02 at 50, 100, 200, 400 g(a.i.)/ha; four single-agent treatments with a single agent of atrazine at 400, 800, 1200, 1600 g(a.i.)/ha, and 20 treatments with mixed formulations at a dosage of 900 g(a.i.)/ha, in which two single agents were mixed in different proportions; and a blank control (spraying water). Each treatment was repeated 4 times. The overground part of all weeds in each treatment was cut 21 days after the treatment, and the fresh weight was weighed to calculate the controlling effect represented by fresh weight. The combined effect by mixing two agents was evaluated by the Gowing method. The theoretical and measured controlling effects (represented by fresh weight) of the two single agents mixed in different proportions were E and E₀ respectively. E=X+Y−XY/100, where X and Y represent the measured controlling effects represented by fresh weight of the two single agents, respectively. The combined effect was additive effect when −5.0%≤E₀−E≤5.0%, synergistic effect when E₀−E>5.0%, and antagonistic effect when E₀−E<−5.0%.

TABLE 3
Synergistic effect as achieved by mixing A02 and atrazine in different
proportions for controlling green bristlegrass
A02 + Atrazine, 900 g(a.i.)/ha

| Proportions | 20 + 1 | 19 + 2 | 18 + 3 | 17 + 4 | 16 + 5 | 15 + 6 | 14 + 7 | 13 + 8 | 12 + 9 | 11 + 10 |
|---|---|---|---|---|---|---|---|---|---|---|
| E₀ − E | 5.6% | 7.2% | 7.5% | 8.0% | 8.6% | 8.5% | 9.3% | 9.2% | 9.6% | 9.6% |
| Proportions | 10 + 11 | 9 + 12 | 8 + 13 | 7 + 14 | 6 + 15 | 5 + 16 | 4 + 17 | 3 + 18 | 2 + 19 | 1 + 20 |
| E₀ − E | 10.0% | 11.6% | 11.3% | 11.8% | 12.0% | 14.0% | 17.6% | 16.2% | 9.3% | 5.0% |

The results in Table 3 showed that the mixture of A02 and atrazine has a significant synergistic effect on the controlling effect of green bristlegrass. When the proportions of A02 and atrazine were 4+17, E₀−E=17.6%, and when the proportions of A02 and atrazine were 3+18, E₀−E=16.2%, which showed an obvious synergistic effect.

### Example 14: Herbicidal Activity Test for Indoor Pre-Emergence Soil Treatment

The herbicidal activity test for indoor pre-emergence soil treatment was performed with some of the N-benzylbenzamide-based compounds of general formula I or II with reference to the method of NY/T 1155.3-2006. Neutral sandy loam soil having an organic content of ≤2% was added to fill ⅘ of the pot, and the soil was completely moistened by infiltrating irrigation from the bottom of the pot. The seeds of the test weed were evenly spread on the soil surface, and then covered by 0.5 cm to 2.0 cm of soil depending on the seed size. Representative compounds in the examples were used to prepare emulsifiable concentrates (10%), and spray treatments to the soil were performed 24 hours after the sowing with the test agents diluted with water, at a spraying dosage of 150 g(a.i.)/ha. Then, the results were investigated by visual inspection 21 days after the treatments. The herbicidal activity of the agent was evaluated on a scale of 1-9 levels according to the symptoms and severity of damage to the test weeds.

Level 1: weed free;

Level 2: equivalent to 0 to 2.5% of the weed in the blank control area;

Level 3: equivalent to 2.6 to 5% of the weed in the blank control area;

Level 4: equivalent to 5.1 to 10% of the weed in the blank control area;

Level 5: equivalent to 10.1 to 15% of the weed in the blank control area;

Level 6: equivalent to 15.1 to 25% of the weed in the blank control area;

Level 7: equivalent to 25.1 to 35% of the weed in the blank control area;

Level 8: equivalent to 35.1 to 67.5% of the weed in the blank control area; and

Level 9: equivalent to 67.6 to 100% of the weed in the blank control area.

Table 4 shows the pre-emergence herbicidal activities of the test compounds against the weeds.

TABLE 4
Weed control effects of the pre-emergence soil treatments of
some of the N-benzylbenzamide-based compounds (150 g a.i./ha)

| | Herbicidal Activity | | | | | |
|---|---|---|---|---|---|---|
| | Barnyard | Green | Digitaria | Amaranthus | | Humulus |
| Compound | grass | bristlegrass | sanguinalis | retroflexus | Piemarker | scandens |
| A01 | 1 | 1 | 1 | 3 | 3 | 4 |
| A02 | 1 | 2 | 1 | 3 | 2 | 4 |
| A03 | 1 | 1 | 2 | 1 | 1 | 2 |
| A07 | 1 | 2 | 1 | 1 | 1 | 3 |
| A09 | 1 | 2 | 2 | 2 | 1 | 3 |
| A14 | 2 | 1 | 2 | 2 | 1 | 3 |
| A22 | 1 | 2 | 1 | 2 | 2 | 4 |
| A23 | 1 | 1 | 1 | 2 | 2 | 4 |
| A24 | 1 | 1 | 1 | 1 | 1 | 1 |
| A28 | 1 | 1 | 1 | 1 | 1 | 2 |
| A29 | 1 | 1 | 2 | 1 | 1 | 2 |
| A35 | 1 | 1 | 2 | 1 | 1 | 2 |
| A85 | 1 | 1 | 1 | 2 | 1 | 3 |
| A87 | 2 | 1 | 2 | 2 | 2 | 3 |
| A89 | 1 | 1 | 2 | 1 | 1 | 2 |
| A91 | 1 | 1 | 2 | 1 | 1 | 2 |
| A92 | 1 | 1 | 1 | 1 | 1 | 2 |
| A94 | 1 | 1 | 1 | 1 | 1 | 2 |
| B01 | 2 | 1 | 1 | 1 | 2 | 2 |
| B02 | 1 | 1 | 2 | 1 | 2 | 2 |
| B07 | 1 | 1 | 1 | 2 | 1 | 3 |
| B08 | 2 | 2 | 2 | 1 | 2 | 2 |
| B14 | 1 | 1 | 2 | 2 | 1 | 3 |
| B15 | 1 | 2 | 1 | 2 | 1 | 3 |
| B22 | 1 | 1 | 1 | 1 | 1 | 2 |
| B27 | 1 | 1 | 1 | 1 | 1 | 1 |
| B30 | 1 | 1 | 1 | 1 | 1 | 2 |
| Isoxaflutole | 1 | 1 | 1 | 1 | 1 | 1 |

The data in Table 4 showed that the test compounds had a good controlling effect on both monocotyledonous weeds and dicotyledonous weeds at a dosage of 150 g(a.i.)/ha, and after the weeds emerged, the seedlings showed albino symptoms, and then gradually withered and died. In addition, the better the light and temperature conditions, the better the effect.

### Example 15: Herbicidal Activity Test for Indoor Post-Emergence Spraying on Stems and Leaves

The post-emergence herbicidal activity test was performed with some of the N-benzylbenzamide-based compounds of general formula I or II with reference to the method of NY/T 1155.4-2006. Neutral sandy loam soil having an organic content of 3% was added to fill ⅘ of the pot, and the soil was completely moistened by infiltrating irrigation from the bottom of the pot. The seeds of the test weed were evenly spread on the soil surface, and then covered by 0.5 cm to 2.0 cm of soil depending on the seed size. After the sowing, the weed was moved to a greenhouse for regular cultivation. Representative compounds in the examples were used to prepare emulsifiable concentrates (10%), and spray treatments were performed at a dosage of 150 g(a.i.)/ha in the stage where 2 leaves and 1 bud were grown for monocotyledonous weeds and the stage where 3 to 5 true leaves were grown for broadleaf weeds. Then, the results were investigated by visual inspection 21 days after the treatments. The herbicidal activity of the agent was evaluated on a scale of 1-9 levels according to the symptoms and severity of damage to the test weeds.

Level 1: all dead;

Level 2: equivalent to 0 to 2.5% of the weed in the blank control area;

Level 3: equivalent to 2.6 to 5% of the weed in the blank control area;

Level 4: equivalent to 5.1 to 10% of the weed in the blank control area;

Level 5: equivalent to 10.1 to 15% of the weed in the blank control area;

Level 6: equivalent to 15.1 to 25% of the weed in the blank control area;

Level 7: equivalent to 25.1 to 35% of the weed in the blank control area;

Level 8: equivalent to 35.1 to 67.5% of the weed in the blank control area; and

Level 9: equivalent to 67.6 to 100% of the weed in the blank control area.

Table 5 shows the post-emergence herbicidal activities of the test compounds against the weeds.

TABLE 5
Weed control effects of post-emergence soil treatments with
some of the N-benzylbenzamide-based compounds (150 g a.i./ha)

| | Herbicidal Activity | | | | | |
|---|---|---|---|---|---|---|
| | Barnyard | Green | Digitaria | Amaranthus | | Humulus |
| Compound | grass | bristlegrass | sanguinalis | retroflexus | Piemarker | scandens |
| A01 | 1 | 1 | 1 | 1 | 1 | 1 |
| A02 | 1 | 2 | 1 | 1 | 1 | 1 |
| A03 | 1 | 1 | 2 | 1 | 1 | 1 |
| A07 | 1 | 2 | 1 | 1 | 1 | 1 |
| A09 | 1 | 2 | 1 | 1 | 1 | 1 |
| A14 | 2 | 1 | 1 | 1 | 1 | 1 |
| A22 | 1 | 2 | 1 | 1 | 1 | 1 |
| A23 | 1 | 1 | 1 | 1 | 1 | 1 |
| A24 | 1 | 1 | 1 | 1 | 1 | 1 |
| A28 | 1 | 1 | 1 | 1 | 1 | 1 |
| A29 | 1 | 1 | 1 | 1 | 1 | 1 |
| A35 | 1 | 1 | 1 | 1 | 1 | 1 |
| A85 | 1 | 1 | 1 | 1 | 1 | 1 |
| A87 | 2 | 1 | 1 | 1 | 1 | 1 |
| A89 | 1 | 1 | 1 | 1 | 1 | 1 |
| A91 | 1 | 1 | 1 | 1 | 1 | 1 |
| A92 | 1 | 1 | 1 | 1 | 1 | 1 |
| A94 | 1 | 1 | 1 | 1 | 1 | 1 |
| B01 | 2 | 1 | 1 | 1 | 1 | 1 |
| B02 | 1 | 1 | 1 | 1 | 1 | 1 |
| B07 | 1 | 1 | 1 | 1 | 1 | 1 |
| B08 | 1 | 2 | 1 | 1 | 1 | 1 |
| B14 | 1 | 1 | 1 | 1 | 1 | 1 |
| B15 | 1 | 1 | 1 | 1 | 1 | 1 |
| B22 | 1 | 1 | 1 | 1 | 1 | 1 |
| B27 | 1 | 1 | 1 | 1 | 1 | 1 |
| B30 | 1 | 1 | 1 | 1 | 1 | 1 |
| Mesotrione | 1 | 1 | 1 | 1 | 1 | 1 |

The data in Table 5 showed that the test compounds had a good controlling effect on both monocotyledonous weeds and dicotyledonous weeds at a dosage of 150 g(a.i.)/ha, and the weeds exhibited characteristic albinism symptoms caused by the pigment synthesis inhibitors 1 to 3 days after the treatment, and then gradually withered and died. In addition, the better the light and temperature conditions, the better the effect.

### Example 16: Post-Emergence Herbicidal Activity Test of Some of the Compounds of General Formula I or II in a Paddy Field

The post-emergence herbicidal activity test was performed in a paddy field with some of the N-benzylbenzamide-based compounds of general formula I or II with reference to the method of GB/T 17980.40-2000. The soil texture of the test paddy field was medium soil, which had an organic content of 2.0% and a pH value of 6.5 in the cultivation layer. Spray treatments were performed in the stage where 4 leaves to 5 leaves were grown for the rice seedlings which was about 1 month after the sowing. Representative compounds in the examples were used to prepare emulsifiable concentrates (10%), and spray treatments were performed with the concentrates in water at a dosage of 150 g(a.i.)/ha, and repeated 4 times for each treatment, and the area was 50 m². 25 g/L of penoxsulam suspension was used as a control agent and applied at a dosage of 250 g(a.i.)/h. The results were investigated by a value estimating survey method 15 days and 30 days after the treatments. The relative weed population quantities were estimated by comparing each agent treated area with an adjacent blank control area, and the results of which were investigated with the following grading standard method using the overall coverage of the weed population as an indicator.

Level 1: weed free;

Level 2: equivalent to 0 to 2.5% of the weed in the blank control area;

Level 3: equivalent to 2.6 to 5% of the weed in the blank control area;

Level 4: equivalent to 5.1 to 10% of the weed in the blank control area;

Level 5: equivalent to 10.1 to 15% of the weed in the blank control area;

Level 6: equivalent to 15.1 to 25% of the weed in the blank control area;

Level 7: equivalent to 25.1 to 35% of the weed in the blank control area;

Level 8: equivalent to 35.1 to 67.5% of the weed in the blank control area; and

Level 9: equivalent to 67.6 to 100% of the weed in the blank control area.

Table 6 shows the post-emergence herbicidal activities of the test compounds against the weeds.

TABLE 6
Post-emergence weed control effects of some of the N-benzylbenzamide-
based compounds against weeds in the paddy field (150 g a.i./ha)

| | Herbicidal activity | | | | | |
|---|---|---|---|---|---|---|
| | 15 days after treatment | | | 30 days after treatment | | |
| | | Broadleaf | | | Broadleaf | |
| Compound | Gramineae | weed | Cyperaceae | Gramineae | weed | Cyperaceae |
| A01 | 2 | 1 | 1 | 2 | 2 | 2 |
| A02 | 1 | 1 | 1 | 1 | 1 | 1 |
| A03 | 2 | 1 | 2 | 1 | 2 | 1 |
| A07 | 2 | 2 | 1 | 2 | 1 | 2 |
| A09 | 2 | 2 | 1 | 2 | 2 | 2 |
| A14 | 1 | 1 | 1 | 2 | 2 | 2 |
| A22 | 1 | 2 | 2 | 1 | 1 | 1 |
| A23 | 1 | 1 | 1 | 1 | 1 | 1 |
| A24 | 1 | 1 | 1 | 1 | 1 | 1 |
| A28 | 2 | 1 | 2 | 1 | 1 | 1 |
| A29 | 2 | 1 | 1 | 1 | 1 | 2 |
| A35 | 2 | 1 | 1 | 1 | 2 | 2 |
| A85 | 2 | 1 | 1 | 1 | 1 | 1 |
| A87 | 2 | 1 | 1 | 1 | 1 | 1 |
| A89 | 1 | 1 | 1 | 1 | 1 | 1 |
| A91 | 2 | 1 | 1 | 2 | 1 | 1 |
| A92 | 2 | 1 | 1 | 2 | 1 | 1 |
| A94 | 2 | 1 | 1 | 2 | 1 | 2 |
| B01 | 2 | 1 | 2 | 2 | 2 | 2 |
| B02 | 2 | 1 | 1 | 2 | 1 | 1 |
| B07 | 2 | 1 | 1 | 2 | 1 | 1 |
| B08 | 2 | 2 | 1 | 2 | 1 | 1 |
| B14 | 1 | 1 | 1 | 1 | 1 | 1 |
| B15 | 2 | 1 | 1 | 1 | 1 | 2 |
| B22 | 1 | 1 | 1 | 1 | 1 | 1 |
| B27 | 1 | 1 | 1 | 1 | 1 | 1 |
| B30 | 1 | 1 | 1 | 1 | 1 | 2 |
| Penoxsulam | 2 | 2 | 2 | 1 | 1 | 2 |

The data in Table 6 showed that the test compounds in the table had a good controlling effect on weeds in the paddy field. The controlling effects 15 days after performing the treatments with compounds A02, A14, A23, A24, A89, B14, B22, B27, B30 on the gramineous weed, broadleaf weed and cyperaceous weed all reached level 1.

### Example 17: Field Test for Weed Control in a Corn Field with Some of the Compounds of General Formula I or II

The weed control efficacy field test was performed in a corn field with some of the N-benzylbenzamide-based compounds of general formula I or II with reference to the method of GB/T 17980.42-2000. The soil texture of the test corn field was sandy loam soil, which had an organic content of 2.0% and a pH value of 6.5 in the cultivation layer. Spray treatments were performed in the stage where 2 leaves to 5 leaves were grown for the corns after the sowing. Representative compounds in the examples were used to prepare emulsifiable concentrates (10%), and spray treatments were performed with the concentrates in water at a dosage of 150 g(a.i.)/ha, and repeated 4 times for each treatment, and the area was 20 m². Oil dispersion with 25% of mesotrione was used as a control agent and applied at a dosage of 150 g(a.i.)/h. The results were investigated by visual inspection 21 days after the treatments. The herbicidal activities of the agents were evaluated on a scale of 1-9 levels according to the symptoms and severity of damage to the test weeds.

Level 1: weed free;

Level 2: equivalent to 0 to 2.5% of the weed in the blank control area;

Level 3: equivalent to 2.6 to 5% of the weed in the blank control area;

Level 4: equivalent to 5.1 to 10% of the weed in the blank control area;

Level 5: equivalent to 10.1 to 15% of the weed in the blank control area;

Level 6: equivalent to 15.1 to 25% of the weed in the blank control area;

Level 7: equivalent to 25.1 to 35% of the weed in the blank control area;

Level 8: equivalent to 35.1 to 67.5% of the weed in the blank control area; and

Level 9: equivalent to 67.6 to 100% of the weed in the blank control area.

Table 7 shows the herbicidal activity of each compound.

TABLE 7
Post-emergence weed control effects of some of the N-benzylbenzamide-based compounds against weeds in the corn field (150 g a.i./ha)

| | 30 days after treatment (%) | | | | | | | | | | |
|---|---|---|---|---|---|---|---|---|---|---|---|
| | Green | Barn- | | | | | | | | | |
| | bristle- | yard | Goose | Digitaria | Chenopodium | Portulaca | Amaranthus | Solanum | Convolvulus | Acalypha | Nutgrass |
| Compound | grass | grass | grass | sanguinalis | album | oleracea | retroflexus | nigrum | arvensis | australis | flatsedge |
| A01 | 1 | 2 | 2 | 2 | 1 | 1 | 1 | 2 | 1 | 2 | 2 |
| A02 | 1 | 1 | 1 | 1 | 1 | 1 | 1 | 1 | 1 | 1 | 1 |
| A03 | 2 | 1 | 2 | 1 | 1 | 1 | 1 | 1 | 1 | 2 | 2 |
| A07 | 2 | 2 | 2 | 2 | 1 | 1 | 1 | 2 | 1 | 2 | 2 |
| A09 | 2 | 2 | 1 | 2 | 1 | 1 | 1 | 2 | 2 | 2 | 2 |
| A14 | 1 | 1 | 1 | 2 | 1 | 1 | 1 | 2 | 1 | 2 | 2 |
| A22 | 2 | 2 | 2 | 1 | 1 | 1 | 1 | 2 | 1 | 2 | 2 |
| A23 | 1 | 2 | 2 | 2 | 1 | 1 | 1 | 2 | 1 | 2 | 3 |
| A24 | 1 | 1 | 2 | 2 | 1 | 1 | 1 | 2 | 1 | 3 | 3 |
| A28 | 1 | 1 | 2 | 2 | 1 | 1 | 1 | 2 | 1 | 2 | 2 |
| A29 | 2 | 1 | 2 | 2 | 1 | 1 | 1 | 2 | 2 | 1 | 2 |
| A35 | 2 | 1 | 2 | 2 | 1 | 1 | 1 | 2 | 2 | 2 | 2 |
| A85 | 1 | 1 | 2 | 2 | 1 | 1 | 1 | 2 | 1 | 1 | 2 |
| A87 | 1 | 1 | 2 | 2 | 1 | 1 | 1 | 2 | 1 | 2 | 2 |
| A89 | 1 | 1 | 2 | 2 | 1 | 1 | 1 | 2 | 1 | 1 | 2 |
| A91 | 2 | 1 | 2 | 2 | 1 | 1 | 1 | 2 | 1 | 2 | 2 |
| A92 | 2 | 1 | 1 | 2 | 1 | 1 | 1 | 2 | 1 | 2 | 2 |
| A94 | 2 | 1 | 1 | 2 | 1 | 1 | 1 | 2 | 2 | 2 | 3 |
| B01 | 1 | 1 | 1 | 1 | 1 | 1 | 1 | 2 | 2 | 2 | 2 |
| B02 | 1 | 1 | 2 | 2 | 1 | 1 | 1 | 2 | 1 | 2 | 2 |
| B07 | 2 | 1 | 2 | 2 | 1 | 1 | 1 | 2 | 1 | 2 | 2 |
| B08 | 1 | 1 | 1 | 1 | 1 | 1 | 1 | 1 | 1 | 1 | 1 |
| B14 | 1 | 1 | 1 | 1 | 1 | 1 | 1 | 1 | 1 | 1 | 1 |
| B15 | 2 | 1 | 2 | 2 | 1 | 1 | 1 | 1 | 2 | 1 | 2 |
| B22 | 1 | 1 | 2 | 2 | 1 | 1 | 1 | 2 | 1 | 1 | 2 |
| B27 | 1 | 1 | 1 | 1 | 1 | 1 | 1 | 1 | 1 | 1 | 1 |
| B30 | 1 | 1 | 1 | 1 | 1 | 1 | 1 | 1 | 1 | 1 | 1 |
| Mesotrione | 1 | 1 | 2 | 2 | 1 | 1 | 1 | 1 | 1 | 2 | 1 |

The data in Table 7 showed that the test compounds in the table had a good controlling effect on weeds in the corn field. The controlling effects 21 days after treatments of the compounds A02, B08, B14, B27, and B30 on green bristlegrass, barnyard grass, goosegrass, Digitaria sanguinalis, Chenopodium album, Portulaca oleracea, Amaranthus retroflexus, Solanum nigrum, Convolvulus arvensis, Acalypha australis and nutgrass flatsedge all reached level 1.

### Example 18: Field Test for Weed Control in a Corn Field with Some of the Compounds of General Formula I or II Tank-Mixed with Atrazine

The weed control efficacy field test was performed in a corn field with some of the N-benzylbenzamide-based compounds of general formula I or II tank-mixed with atrazine with reference to the method of GB/T 17980.42-2000. The soil texture of the test corn field was sandy loam soil, which had an organic content of 2.0% and a pH value of 6.5 in the cultivation layer. Spray treatments were performed in the stage where 2 leaves to 5 leaves were grown for the corns after the sowing. Representative compounds in the examples were used to prepare emulsifiable concentrates (10%), tank-mixed with a suspension with 40% of atrazine for secondary absorption, and then spray treatments were performed with the mixture in water, and repeated 4 times for each treatment, and the area was 20 m². The suspension with 40% of atrazine was used as a control agent and applied at a dosage of 600 g(a.i.)/h. The results were investigated by visual inspection 21 days after the treatments. The herbicidal activities of the agents were evaluated on a scale of 1-9 levels according to the symptoms and severity of damage to the test weeds. Table 8 shows the herbicidal activity of each compound.

TABLE 8
Post-emergence weed control effects in the corn field of some of the compounds of general formula I or II tank-mixed with atrazine

| | | Herbicidal activity (%) | | | | | | | | | |
|---|---|---|---|---|---|---|---|---|---|---|---|
| | | Barn- | Green | | | | | | | | |
| | Dosage | yard | bristle- | Digitaria | Goose | Chenopodium | Portulaca | Amaranthus | Solanum | Pie | Nutgrass |
| Treatment | g (a.i.)/ha | grass | grass | sanguinalis | grass | album | oleracea | retroflexus | nigrum | marker | flatsedge |
| A01 + atrazine | 75 + 375 | 1 | 1 | 1 | 2 | 1 | 1 | 1 | 1 | 1 | 1 |
| A02 + atrazine | 100 + 350 | 1 | 1 | 1 | 1 | 1 | 1 | 1 | 1 | 1 | 1 |
| A03 + atrazine | 150 + 300 | 1 | 1 | 1 | 2 | 1 | 2 | 1 | 1 | 1 | 1 |
| A07 + atrazine | 75 + 375 | 1 | 1 | 1 | 1 | 1 | 1 | 1 | 1 | 1 | 1 |
| A09 + atrazine | 75 + 375 | 1 | 1 | 1 | 1 | 1 | 1 | 1 | 1 | 1 | 1 |
| A14 + atrazine | 150 + 300 | 1 | 1 | 1 | 1 | 1 | 1 | 1 | 1 | 1 | 1 |
| A22 + atrazine | 100 + 350 | 1 | 1 | 1 | 1 | 1 | 1 | 1 | 1 | 1 | 1 |
| A23 + atrazine | 100 + 350 | 1 | 1 | 1 | 1 | 1 | 1 | 1 | 1 | 1 | 1 |
| A24 + atrazine | 75 + 375 | 1 | 1 | 1 | 1 | 1 | 1 | 1 | 1 | 1 | 1 |
| A28 + atrazine | 100 + 350 | 1 | 1 | 1 | 1 | 1 | 1 | 1 | 1 | 1 | 1 |
| A29 + atrazine | 100 + 350 | 1 | 1 | 1 | 1 | 1 | 1 | 1 | 1 | 1 | 1 |
| A35 + atrazine | 150 + 300 | 1 | 1 | 1 | 1 | 1 | 1 | 1 | 1 | 1 | 1 |
| A85 + atrazine | 150 + 300 | 1 | 1 | 1 | 1 | 1 | 1 | 1 | 1 | 1 | 1 |
| A87 + atrazine | 150 + 300 | 1 | 1 | 1 | 1 | 1 | 1 | 1 | 1 | 1 | 1 |
| A89 + atrazine | 75 + 375 | 1 | 1 | 1 | 1 | 1 | 1 | 1 | 1 | 1 | 1 |
| A91 + atrazine | 75 + 375 | 1 | 1 | 1 | 1 | 1 | 1 | 1 | 1 | 1 | 1 |
| A92 + atrazine | 75 + 375 | 1 | 1 | 1 | 1 | 1 | 1 | 1 | 1 | 1 | 1 |
| A94 + atrazine | 150 + 300 | 1 | 1 | 1 | 1 | 1 | 1 | 1 | 1 | 1 | 1 |
| B01 + atrazine | 150 + 300 | 1 | 1 | 1 | 1 | 1 | 1 | 1 | 1 | 1 | 1 |
| B02 + atrazine | 150 + 300 | 1 | 1 | 1 | 1 | 1 | 1 | 1 | 1 | 1 | 1 |
| B07 + atrazine | 75 + 375 | 1 | 1 | 1 | 1 | 1 | 1 | 1 | 1 | 1 | 1 |
| B08 + atrazine | 75 + 375 | 1 | 1 | 1 | 1 | 1 | 1 | 1 | 1 | 1 | 1 |
| B14 + atrazine | 100 + 350 | 1 | 1 | 1 | 1 | 1 | 1 | 1 | 1 | 1 | 1 |
| B15 + atrazine | 100 + 350 | 1 | 1 | 1 | 1 | 1 | 1 | 1 | 1 | 1 | 1 |
| B22 + atrazine | 100 + 350 | 1 | 1 | 1 | 1 | 1 | 1 | 1 | 1 | 1 | 1 |
| B27 + atrazine | 75 + 375 | 1 | 1 | 1 | 1 | 1 | 1 | 1 | 1 | 1 | 1 |
| B30 + atrazine | 75 + 375 | 1 | 1 | 1 | 1 | 1 | 1 | 1 | 1 | 1 | 1 |
| 40% of atrazine | 600 | 4 | 4 | 4 | 4 | 1 | 1 | 1 | 1 | 1 | 3 |
| suspension | | | | | | | | | | | |

The data in Table 8 showed that the compounds of general formula I or II tank-mixed with atrazine in a certain ratio had controlling effects of up to level 1 on the weeds in the corn filed, such as green bristlegrass, barnyard grass, goosegrass, Digitaria sanguinalis, Chenopodium album, Portulaca oleracea, Amaranthus retroflexus, Solanum nigrum, piemarker and nutgrass flatsedge. The suspension having 40% of atrazine used at a dosage of 600 g(a.i.)/ha had a controlling effect of up to level 1 on broadleaf grasses, but had a poor controlling effect on gramineous weeds and nutgrass flatsedge. The compound of general formula I or II tank-mixed with atrazine in a certain ratio significantly improved the controlling effects on gramineous weeds and nutgrass flatsedge, and even had a controlling effects of up to level 1 on older gramineous weeds and nutgrass flatsedge, which are basically resistant to atrazine.

### Example 19: Field Test for Weed Control in a Corn Field with Mixed Formulations Including Some of the Compounds of General Formula I or II and Atrazine

The post-emergence weed control effect field test was performed in a corn field with mixed formulations of some of the compounds of general formula I or II and atrazine with reference to the method of NY/T 1155.4-2006. Post-emergence stem and leaf spraying treatments were performed in the stage where 4 leaves were grown for the corns at a dosage of 30 kg per mu. The results were investigated by visual inspection 21 days after the treatments. The herbicidal activities of the agents were evaluated on a scale of 1-9 levels according to the symptoms and severity of damage to the test weeds. The suspension with 40% of atrazine was used as a control agent and applied at a dosage of 600 g(a.i.)/h. Table 9 shows the herbicidal activities.

TABLE 9
Post-emergence weed control effect in the com field of the mixed formulations
including some of the compounds of general formula I or II and atrazine

| | | Herbicidal activity (%) | | | | | | | | | |
|---|---|---|---|---|---|---|---|---|---|---|---|
| | | Barn- | Green | | | | | | | | |
| | Dosage | yard | bristle- | Digitaria | Goose | Chenopodium | Portulaca | Amaranthus | Solanum | Pie | Nutgrass |
| Treatment | g (a.i.)/ha | grass | grass | sanguinalis | grass | album | oleracea | retroflexus | nigrum | marker | flatsedge |
| A01 + atrazine oil | 75 + 375 | 1 | 1 | 1 | 1 | 1 | 1 | 1 | 1 | 1 | 1 |
| dispersion | | | | | | | | | | | |
| A02 + atrazine oil | 100 + 350 | 1 | 1 | 1 | 1 | 1 | 1 | 1 | 1 | 1 | 1 |
| dispersion | | | | | | | | | | | |
| A03 + atrazine oil | 150 + 300 | 1 | 1 | 1 | 2 | 1 | 2 | 1 | 1 | 1 | 1 |
| dispersion | | | | | | | | | | | |
| A07 + atrazine oil | 75 + 375 | 1 | 1 | 1 | 1 | 1 | 1 | 1 | 1 | 1 | 1 |
| dispersion | | | | | | | | | | | |
| A09 + atrazine oil | 75 + 375 | 1 | 1 | 1 | 1 | 1 | 1 | 1 | 1 | 1 | 1 |
| dispersion | | | | | | | | | | | |
| A14 + atrazine oil | 150 + 300 | 1 | 1 | 1 | 1 | 1 | 1 | 1 | 1 | 1 | 1 |
| dispersion | | | | | | | | | | | |
| A22 + atrazine oil | 100 + 350 | 1 | 1 | 1 | 1 | 1 | 1 | 1 | 1 | 1 | 1 |
| dispersion | | | | | | | | | | | |
| A23 + atrazine oil | 100 + 350 | 1 | 1 | 1 | 1 | 1 | 1 | 1 | 1 | 1 | 1 |
| dispersion | | | | | | | | | | | |
| A24 + atrazine oil | 75 + 375 | 1 | 1 | 1 | 1 | 1 | 1 | 1 | 1 | 1 | 1 |
| dispersion | | | | | | | | | | | |
| A28 + atrazine oil | 100 + 350 | 1 | 1 | 1 | 1 | 1 | 1 | 1 | 1 | 1 | 1 |
| dispersion | | | | | | | | | | | |
| A29 + atrazine oil | 100 + 350 | 1 | 1 | 1 | 1 | 1 | 1 | 1 | 1 | 1 | 1 |
| dispersion | | | | | | | | | | | |
| A35 + atrazine oil | 150 + 300 | 1 | 1 | 1 | 1 | 1 | 1 | 1 | 1 | 1 | 1 |
| dispersion | | | | | | | | | | | |
| A85 + atrazine oil | 150 + 300 | 1 | 1 | 1 | 1 | 1 | 1 | 1 | 1 | 1 | 1 |
| dispersion | | | | | | | | | | | |
| A87 + atrazine oil | 150 + 300 | 1 | 1 | 1 | 1 | 1 | 1 | 1 | 1 | 1 | 1 |
| dispersion | | | | | | | | | | | |
| A89 + atrazine oil | 75 + 375 | 1 | 1 | 1 | 1 | 1 | 1 | 1 | 1 | 1 | 1 |
| dispersion | | | | | | | | | | | |
| A91 + atrazine oil | 75 + 375 | 1 | 1 | 1 | 1 | 1 | 1 | 1 | 1 | 1 | 1 |
| dispersion | | | | | | | | | | | |
| A92 + atrazine oil | 75 + 375 | 1 | 1 | 1 | 1 | 1 | 1 | 1 | 1 | 1 | 1 |
| dispersion | | | | | | | | | | | |
| A94 + atrazine oil | 150 + 300 | 1 | 1 | 1 | 1 | 1 | 1 | 1 | 1 | 1 | 1 |
| dispersion | | | | | | | | | | | |
| B01 + atrazine oil | 150 + 300 | 1 | 1 | 1 | 1 | 1 | 1 | 1 | 1 | 1 | 1 |
| dispersion | | | | | | | | | | | |
| B02 + atrazine oil | 150 + 300 | 1 | 1 | 1 | 1 | 1 | 1 | 1 | 1 | 1 | 1 |
| dispersion | | | | | | | | | | | |
| B07 + atrazine oil | 75 + 375 | 1 | 1 | 1 | 1 | 1 | 1 | 1 | 1 | 1 | 1 |
| dispersion | | | | | | | | | | | |
| B08 + atrazine oil | 75 + 375 | 1 | 1 | 1 | 1 | 1 | 1 | 1 | 1 | 1 | 1 |
| dispersion | | | | | | | | | | | |
| B14 + atrazine oil | 100 + 350 | 1 | 1 | 1 | 1 | 1 | 1 | 1 | 1 | 1 | 1 |
| dispersion | | | | | | | | | | | |
| B15 + atrazine oil | 100 + 350 | 1 | 1 | 1 | 1 | 1 | 1 | 1 | 1 | 1 | 1 |
| dispersion | | | | | | | | | | | |
| B22 + atrazine oil | 100 + 350 | 1 | 1 | 1 | 1 | 1 | 1 | 1 | 1 | 1 | 1 |
| dispersion | | | | | | | | | | | |
| B27 + atrazine oil | 75 + 375 | 1 | 1 | 1 | 1 | 1 | 1 | 1 | 1 | 1 | 1 |
| dispersion | | | | | | | | | | | |
| B30 + atrazine oil | 75 + 375 | 1 | 1 | 1 | 1 | 1 | 1 | 1 | 1 | 1 | 1 |
| dispersion | | | | | | | | | | | |
| 40% of atrazine | 600 | 4 | 4 | 4 | 4 | 1 | 1 | 1 | 1 | 1 | 3 |
| suspension | | | | | | | | | | | |

The data in Table 9 showed that the mixed formulations as prepared by the compounds of general formula I or II and atrazine had controlling effects of up to level 1 at a dosage of 450 g (a.i.)/ha on the weeds in the corn filed, such as green bristlegrass, barnyard grass, goosegrass, Digitaria sanguinalis, Chenopodium album, Portulaca oleracea, Amaranthus retroflexus, Solanum nigrum, piemarker and nutgrass flatsedge.

The use of the N-benzylbenzamide-based compound as a herbicide according to the present disclosure has been described through specific examples. Those skilled in the art may learn from the content of the present disclosure and appropriately change the raw materials, process conditions and other factors to achieve corresponding other purposes. In addition, the related changes do not deviate from the content of the present disclosure, and all similar replacements and changes are obvious to those skilled in the art, and are considered to be included in the scope of the present disclosure.

1. An herbicide comprising a N-benzylbenzamide-based compound of following general formula (I) or (II) or its salt as a component, 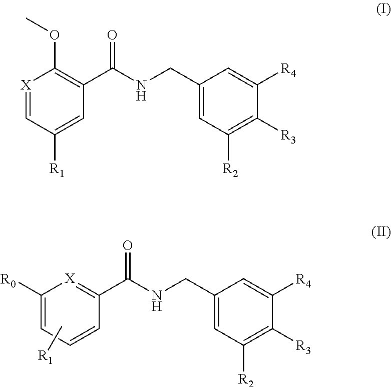
wherein in the general formula (I) or (II),
R₀ is selected from methyl or methoxy;
R₁ is selected from hydrogen, C₁-C₄ alkyl, C₁-C₄ alkoxy, halo C₁-C₄ alkyl, halo C₁-C₄ alkoxy, halogen or cyano;
R₂, R₃ and R₄ are each independently selected from hydrogen, C₁-C₄ alkyl, C₁-C₄ alkoxy, halogen, cyano, halo C₁-C₄ alkyl, halo C₁-C₄alkoxy or methylsulfonyl; and
X is selected from C or N.

2. The herbicide according to claim 1, wherein in the general formula (I) or (II),
R₀ is selected from methyl or methoxy;
R₁ is selected from hydrogen, methyl, methoxy, fluorine, chlorine, bromine or cyano;
R₂, R₃ and R₄ are each independently selected from hydrogen, methyl, methoxy, fluorine, chlorine, bromine, cyano or methylsulfonyl; and
X is selected from C or N.

3. The herbicide according to claim 1, wherein the N-benzylbenzamide-based compound of general formula (I) is prepared by a reaction Scheme I in a suitable solvent and a suitable alkaline condition with a synthetic scheme as follows: 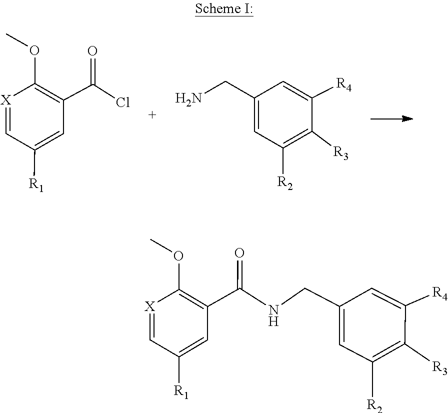
the reaction Scheme I is carried out in a suitable solvent, and the suitable solvent is selected from tetrahydrofuran, acetonitrile, toluene, xylene, N,N-dimethylformamide, dichloromethane, chloroform or pyridine; a suitable alkali is selected from triethylamine, pyridine, potassium carbonate or sodium hydroxide; a reaction temperature is between ice bath and a boiling temperature of the solvent; and a reaction time is 30 minutes to 20 hours.

4. The herbicide according to claim 1, wherein the N-benzylbenzamide-based compound of general formula (II) is prepared by a reaction Scheme II in a suitable solvent and a suitable alkaline condition with a synthetic scheme as follows: 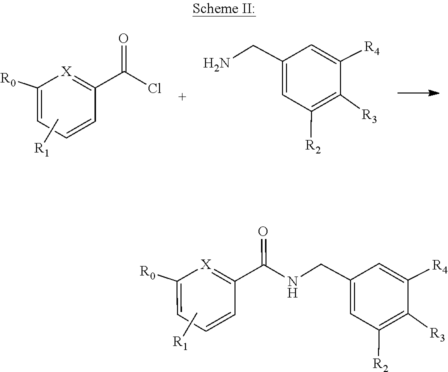
the reaction Scheme II is carried out in a suitable solvent, and the suitable solvent is selected from tetrahydrofuran, acetonitrile, toluene, xylene, N,N-dimethylformamide, dichloromethane, chloroform or pyridine; a suitable alkali is selected from triethylamine, pyridine, potassium carbonate or sodium hydroxide; a reaction temperature is between ice bath and a boiling temperature of the solvent; and a reaction time is 30 minutes to 20 hours.

5. The herbicide according to claim 1, wherein the N-benzylbenzamide-based compound serves as a pigment synthesis inhibitor to control weeds in field crop growing places or non-farming crop places.

6. The use according to claim 5, wherein the weeds comprise: Sinapis, Brassica, Galium, Stellaria, Chenopodium, Kochia, Urtica, Senecio, Amaranthus, Portulaca, Xanthium, Ipomoea, Polygonum, Ambrosia, Cirsium, Sonchus, Solanum, Veronica, Datura, Viola, Papaver, Centaurea, Galinsoga, Rotala, Lindernia, Sesbania, Trifolium, Abutilon, Lamium, Matricaria, Artemisia, Pharbitis, Echinochloa, Setaria, Panicum, Digitaria, Phleum, Poa, Festuca, Lolium, Bromus, Avena, Cyperus, Sorghum, Agropyron, Monochoria, Fimbristylis, Sagittaria, Scirpus, Paspalum, Ischaemum, Agrostis, Alopecurus, Cynodon, Commelina, Brachiaria or Leptochloa.

7. The herbicide according to claim 5, wherein the weeds are barnyard grass, pondweed, Monochoria vaginalis, Cyperus difformis, Scirpus planiculmis, Leptochloa chinensis, Avena fatua, Digitaria sanguinalis, goosegrass, green bristlegrass, Alopecurus aequalis, flixweed, Capsella bursa pastoris, Lepidium apetalum, Humulus scandens, Polygonum aviculare, Convolvulus arvensis, Fallopia convolvulus, Cyperus rotundus, Chenopodium album, Polygonum lapathifolium, Amaranthus retroflexus, Portulaca oleracea, Amaranthus blitum, Cuscuta chinensis, piemarker, Galium spurium, Eclipta prostrata, Xanthium strumarium, bermudagrass, cogon, Solanum nigrum, Myosoton aquaticum, Chromolaena odorata, Ageratum conyzoides, Rotala rotundifolia, Artemisia argyi, dandelion, Erigeron annuus, Hemisteptia lyrata, Artemisia capillaris, Ixeris polycephala, field thistle, setose thistle, Elsholtzia ciliata, woundwort or Acalypha australis.

8. An agricultural herbicide composition, wherein a herbicidal component of the composition is the N-benzylbenzamide-based compound or its salt according to claim 1, and the composition further comprises an agriculturally acceptable carrier.

9. An agricultural herbicide mixed formulation, wherein a herbicidal component of the mixed formulation is the N-benzylbenzamide-based compound or its salt according to claim 1, and the mixed formulation further comprises a herbicidal component serving as an acetyl-CoA carboxylase inhibitor, an acetolactate synthase inhibitor, an auxin herbicide, a carotenoid biosynthesis inhibitor, a photosynthesis inhibitor, or a cell wall biosynthesis inhibitor.

10. The mixed formulation according to claim 9, wherein the herbicidal components in the mixed formulation are the N-benzylbenzamide-based compound or its salt and atrazine in a weight ratio from 1:10 to 10:1.
